(19) United States
(12) Patent Application Publication
(10) Pub. No.: US 2011/0066222 A1
(43) Pub. Date: Mar. 17, 2011

(54) Polymeric Stent and Method of Making Same

(76) Inventors: Yunbing Wang, Sunnyvale, CA (US); Manish B. Gada, Santa Clara, CA (US)

(21) Appl. No.: 12/558,105

(22) Filed: Sep. 11, 2009

(51) Int. Cl. A61F 2/06; B29C 49/08

(52) U.S. Cl. 623/1.15; 264/535

(57) ABSTRACT

A stent may be formed from a PLLA tubular polymer construct that is deformed in a blow mold. A desirable polymer morphology resulting in improved stent performance is obtained with a selected radial axial expansion ratio from about 20% to about 70%, a selected radial expansion ratio from about 400% to about 500%, a selected axial rate of deformation propagation at or about 0.3 mm/minute, a selected expansion pressure at or about 130 psi, and a selected expansion temperature that does not exceed 200 deg F. The tubular polymer construct may also be made of PLGA, PLLA-co-PDLA, PLLD/PDLA stereocomplex, and PLLA-based polyester block copolymer containing a rigid segment of PLLA or PLGA and a soft segment of PCL or PTMC.

### FIELD OF THE INVENTION

This invention relates generally to fabrication of implantable prostheses, more particularly, to fabrication of stents from blow molded polymeric tubes.

### BACKGROUND OF THE INVENTION

Radially expandable endoprostheses are artificial devices adapted to be implanted in an anatomical lumen. An “anatomical lumen” refers to a cavity, duct, of a tubular organ such as a blood vessel, urinary tract, and bile duct. Stents are examples of endoprostheses that are generally cylindrical in shape and function to hold open and sometimes expand a segment of an anatomical lumen. Stents are often used in the treatment of atherosclerotic stenosis in blood vessels. “Stenosis” refers to a narrowing or constriction of the diameter of a bodily passage or orifice. In such treatments, stents reinforce the walls of the blood vessel and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through an anatomical lumen to a desired treatment site, such as a lesion. “Deployment” corresponds to expansion of the stent within the lumen at the treatment region. Delivery and deployment of a stent are accomplished by positioning the stent about one end of a catheter, inserting the end of the catheter through the skin into an anatomical lumen, advancing the catheter in the anatomical lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon prior to insertion in an anatomical lumen. At the treatment site within the lumen, the stent is expanded by inflating the balloon. The balloon may then be deflated and the catheter withdrawn from the stent and the lumen, leaving the stent at the treatment site. In the case of a self-expanding stent, the stent may be secured to the catheter via a retractable sheath. When the stent is at the treatment site, the sheath may be withdrawn which allows the stent to self-expand.

The stent must be able to satisfy a number of functional requirements. The stent must be capable of withstanding the structural loads, namely radial compressive forces, imposed on the stent as it supports the walls of a vessel after deployment. Therefore, a stent must possess adequate radial strength. Radial strength, which is the ability of a stent to resist radial compressive forces, is due to strength and rigidity around a circumferential direction of the stent. After deployment, the stent must also adequately maintain its size and shape throughout its service life despite the various forces that may come to bear on it, including the cyclic loading induced by the beating heart.

In addition to high radial strength, the stent must also possess sufficient toughness so that the stent exhibits sufficient flexibility to allow for crimping on the a delivery device, flexure during delivery through an anatomical lumen, and expansion at the treatment site. Longitudinal flexibility is important to allow the stent to be maneuvered through a tortuous vascular path and to enable it to conform to a deployment site that may not be linear or may be subject to flexure. A stent should have sufficient toughness so that it is resistant to crack formation, particularly, in high strain regions.

Furthermore, it may be desirable for a stent to be made of a biodegradable or bioerodable polymer. In many treatment applications, the presence of a stent in a body may be necessary for a limited period of time until its intended function of, for example, maintaining vascular patency and/or drug delivery is accomplished. Also, it is believed that biodegradable stents allow for improved healing of the anatomical lumen as compared to metal stents, which may lead to a reduced incidence of late stage thrombosis.

However, a potential shortcoming of polymer stents compared to metal stents of the same dimensions, is that polymer stents typically have less radial strength and rigidity. Relatively low radial strength potentially contributes to relatively high recoil of polymer stents after implantation into an anatomical lumen. “Recoil” refers to the undesired retraction of a stent radially inward from its deployed diameter due to radially compressive forces that bear upon it after deployment. Furthermore, another potential problem with polymer stents is that struts can crack or fracture during crimping, delivery and deployment, especially for brittle polymers. Some crystalline or semi-crystalline polymers that may be suitable for use in implantable medical devices generally have potential shortcomings with respect to some mechanical characteristics, in particular, fracture toughness, when used in stents.

Some polymers, such as poly(L-lactide) (“PLLA”), poly(L-lactide-co-glycolide) (“PLGA”), poly(L-lactide-co-D-lactide) (“PLLA-co-PDLA”) with less than 10% D-lactide, and PLLD/PDLA stereocomplex, are stiff and strong but can exhibit a brittle fracture mechanism at physiological conditions in which there is little or no plastic deformation prior to failure. Physiological conditions include, but are limited to, human body temperature, approximately 37° C. A stent fabricated from such polymers can have insufficient toughness for the range of use of a stent. As a result, cracks, particularly in high strain regions, can be induced which can result in mechanical failure of the stent.

Accordingly, there is a need for manufacturing methods for fabricating polymeric stents with sufficient radial strength, fracture toughness, low recoil, and sufficient shape stability.

### SUMMARY OF THE INVENTION

Briefly and in general terms, the present invention is directed to a stent and a method of forming a stent.

In aspects of the present invention, a method of forming a stent comprises deforming a precursor tube of poly(L-lactide) to form a deformed tube. The deforming includes maintaining fluid pressure in the tube at a process pressure from about 110 psi to about 150 psi, heating the tube to a process temperature from about 160 deg F. to about 220 deg F., radially expanding the precursor tube according to a radial expansion ratio between about 300% and about 450% during the maintaining of fluid pressure and the heating, and axially extending the precursor tube according to an axial extension ratio from about 20% to about 100% during the maintaining of fluid pressure and the heating. The method further comprises forming a network of stent struts from the deformed tube. In detailed aspects of the present invention, heating the tube includes heating a tubular mold containing the tube, the heating including moving a heat source disposed outside the tube at a linear rate of movement parallel to the central axis of the mold, the linear rate of movement being about 0.1 mm to 0.7 mm per minute. In further aspects of the present invention, a stent comprises the network of stent struts formed from the deformed tube.

In aspects of the present invention, a method of making a stent comprises providing a poly(L-lactide) tube inside a tubular mold, heating a segment of the tube with a heat source, the segment of the tube being heated to a process temperature from about 160 deg F. to about 220 deg F., and moving the heat source in a process direction. The method further comprises causing deformation of the heated segment to form a deformed segment of the tube, the deformation propagating in the process direction, the deformation including radial expansion and axial extension of the tube, the radial expansion in accordance with a radial expansion ratio between about 300% and about 450%, the axial extension in accordance with an axial extension ratio between about 20% and about 100%. The method further comprises forming stent struts from the deformed segment.

A method for making a stent, according to aspects of the present invention, comprises deforming a precursor tube of a polymer formulation to form a deformed tube. The deforming includes maintaining fluid pressure in the tube at a process pressure from about 50 psi to about 200 psi, heating the tube to a process temperature from about 100 deg. F. to about 300 deg F., radially expanding the precursor tube according to a radial expansion ratio between about 100% and about 600% during the maintaining of fluid pressure and the heating, and axially extending the precursor tube according to an axial extension ratio from about 10% to about 200% during the maintaining of fluid pressure and the heating. The method further comprises forming a network of stent struts from the deformed tube. In further aspects, the polymer formation is a material selected from the group consisting of PLGA, PLLA-co-PDLA, PLLD/PDLA stereocomplex, and PLLA-based polyester block copolymer containing a rigid segment and a soft segment, the rigid segment being PLLA or PLGA, the soft segment being PCL or PTMC.

A method of making a stent, according to aspects of the present invention, comprises providing a polymer tube inside a tubular mold, the polymer tube made of a polymer formulation selected from the group consisting of PLGA, PLLA-co-PDLA, PLLD/PDLA stereocomplex, and PLLA-based polyester block copolymer containing a rigid segment and a soft segment, the rigid segment being PLLA or PLGA, the soft segment being PCL or PTMC. The method further comprises heating a segment of the tube with a heat source, the segment of the tube being heated to a process temperature from about 100 deg F. to about 300 deg F. The method further comprises moving the heat source in a process direction. The method further comprises causing deformation of the heated segment to form a deformed segment of the tube, the deformation propagating in the process direction, the deformation including radial expansion and axial extension of the tube, the radial expansion in accordance with a radial expansion ratio between about 100% and about 600%, the axial extension in accordance with an axial extension ratio from about 10% to about 200%. The method further comprises forming stent struts from the deformed segment.

The features and advantages of the invention will be more readily understood from the following detailed description which should be read in conjunction with the accompanying drawings.

### BRIEF DESCRIPTION OF THE DRAWINGS

FIG. 1 is a perspective view of a stent.

FIG. 2 is a perspective view of a polymer tube for making a stent.

FIG. 3A is an axial cross-sectional view of a blow molding system showing a blow mold and a polymer tube in the blow mold.

FIG. 3B is a radial cross-sectional view of the blow molding system of FIG. 3A, showing nozzles heating the blow mold.

FIG. 3C is an axial cross-sectional view of the blow molding system of FIG. 3A, showing the polymer tube being deformed.

FIG. 3D is an axial cross-sectional view of the blow molding system of FIG. 3A, showing further deformation of the polymer tube.

FIG. 4 is a schematic plot of quiescent crystal nucleation rate and the quiescent crystal growth rate, and the overall rate of quiescent crystallization.

FIG. 5 is a top view of a pattern of struts for a stent.

FIG. 6 is a perspective view of a portion of a stent having the pattern of FIG. 5.

### DETAILED DESCRIPTION OF PREFERRED EMBODIMENTS

The various embodiments of the present invention relate to methods of fabricating a polymeric stent that has good or optimal toughness and selected mechanical properties along the axial, radial and circumferential directions. The present invention can be applied to devices including, but is not limited to, self-expandable stents, balloon-expandable stents, stent-grafts, grafts (e.g., aortic grafts), and generally to tubular implantable medical devices.

For the purposes of the present invention, the following terms and definitions apply:

The glass transition temperature (referred to herein as “Tg”) is the temperature at which the amorphous domains of a polymer change from a brittle vitreous state to a solid deformable or ductile state at atmospheric pressure. In other words, Tg corresponds to the temperature where the onset of segmental motion in the chains of the polymer occurs. Tg of a given polymer can be dependent on the heating rate and can be influenced by the thermal history of the polymer. Furthermore, the chemical structure of the polymer heavily influences the glass transition by affecting mobility of polymer chains.

“Stress” refers to force per unit area, as in the force acting through a small area within a plane within a subject material. Stress can be divided into components, normal and parallel to the plane, called normal stress and shear stress, respectively. Tensile stress, for example, is a normal component of stress that leads to expansion (increase in length) of the subject material. In addition, compressive stress is a normal component of stress resulting in compaction (decrease in length) of the subject material.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Modulus” may be defined as the ratio of a component of stress or force per unit area applied to a material divided by the strain along an axis of applied force that results from the applied force. For example, a material has both a tensile and a compressive modulus.

“Toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough is said to be brittle. Brittle materials are strong, but cannot deform very much before breaking.

As used herein, the terms “axial” and “longitudinal” are used interchangeably and refer to a direction, orientation, or line that is parallel or substantially parallel to the central axis of a stent or the central axis of a tubular construct. The term “circumferential” refers to the direction along a circumference of the stent or tubular construct. The term “radial” refers to a direction, orientation, or line that is perpendicular or substantially perpendicular to the central axis of the stent or the central axis of a tubular construct.

Mechanical properties of a polymer may be modified by processes that alter the molecular structure or morphology of the polymer. Polymers in the solid state may be completely amorphous, partially crystalline, or almost completely crystalline. Crystalline regions are where polymer molecules are geometrically arranged in a regular order or pattern. Crystalline regions may be clusters of polymer crystals. Each crystal may have polymer molecules arranged geometrically around a nucleus. Amorphous regions in a polymer matrix are where polymer molecules have no regular order or arrangement. Amorphous regions may be located between ordered polymer chains, between polymer crystals, and between clusters of polymer crystals.

Polymer molecule chains in crystalline regions may radiate outwardly from many nuclei without a preferred orientation or alignment. In other instances, polymer molecules in crystalline regions may have a preferred orientation or long range order with respect to a particular direction, as may occur with strain induced crystallization.

As indicated above, molecular orientation in a polymer may be induced, and hence modify mechanical properties, by applying a stress to the polymer which deforms the polymer in the direction of the applied stress. The degree of molecular orientation induced with applied stress may depend upon the temperature of the polymer. For example, below the glass transition temperature, Tg, of a polymer, polymer segments do not have sufficient energy to move past one another. In general, molecular orientation may not be induced without sufficient segmental mobility. Above Tg, molecular orientation may be induced with applied stress since rotation of polymer chains, and hence segmental mobility is possible. Between Tg and the melting temperature of the polymer (referred to herein as “Tm”), rotational barriers exist, however, the barriers are not great enough to substantially prevent segmental mobility. As the temperature of a polymer is increased above Tg, the energy barriers to rotation decrease and segmental mobility of polymer chains tend to increase. As a result, as the temperature increases, molecular orientation is more easily induced with applied stress. A polymer with a high level of polymer chain alignment would have enhanced strength and toughness in the direction of alignment of the polymer chains.

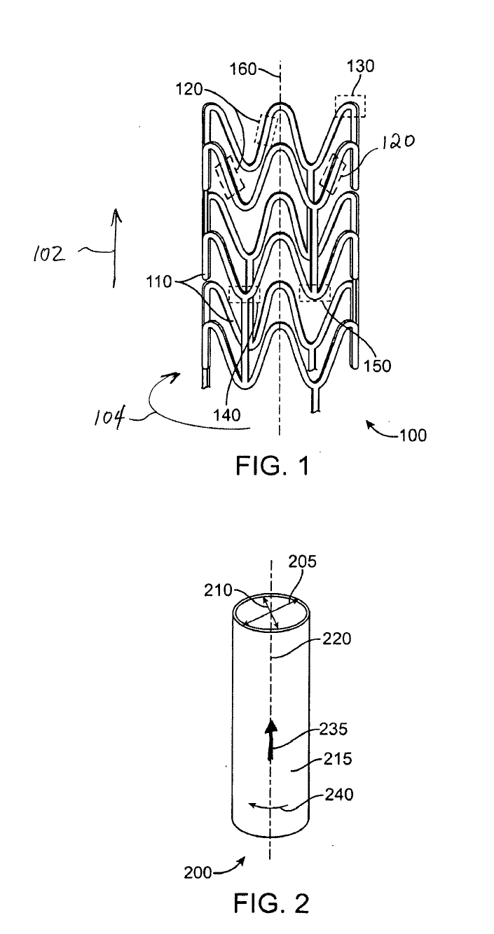

Referring now in more detail to the exemplary drawings for purposes of illustrating embodiments of the invention, wherein like reference numerals designate corresponding or like elements among the several views, there is shown in FIG. 1 a stent 100 in an uncrimped state or a deployed state. The stent 110 has a scaffolding composed of a pattern of interconnected structural elements or struts 110. The struts 110 form a hollow body having cylindrical shape or tubular shape. The struts 110 have straight or relatively straight portions 120. The struts also have bending elements 130, 140, and 150, which are configured to bend during stent crimping and deployment to allow the straight portions 120 to collapse next to each other and expand apart from each other. The tubular body has two opposite open ends, a central passageway that runs from one end to the opposite end, and a central axis 160 that extends longitudinally through the center of the central passageway. Surfaces of the struts 110 that face radially inward toward the central axis 160 form the luminal or inner surface of the stent. Surfaces of the struts 110 that face radially outward away from the central axis 160 form the abluminal or outer surface of the stent. When deployed in a blood vessel, the luminal surface faces blood flowing through the central passageway of the stent and the abluminal surface faces and supports the walls of the blood vessel.

The stresses involved during compression and expansion are generally distributed throughout the various structural elements of the stent pattern. The present invention is not limited to the stent pattern depicted in FIG. 1. The variation in stent patterns is virtually unlimited.

The struts 110, which may serve as the underlying structure or substrate of a stent, is completely or at least in part made from a biodegradable polymer or combination of biodegradable polymers, a biostable polymer or combination of biostable polymers, or a combination of biodegradable and biostable polymers. Suitable examples of polymers include without limitation, poly(L-lactide) (“PLLA”) and poly(lactic-co-glycolic acid) (“PLGA”). PLLA and PLGA are semi-crystalline polymers in that their morphology includes crystalline and amorphous regions, though the amount of crystallinity can be altered. For example, the maximum crystallinity of pure PLLA is about 70%, while that of PLGA with 20% GA is below 10%. Additionally, a polymer-based coating on the stent substrate can be a biodegradable polymer or combination of biodegradable polymers, a biostable polymer or combination of biostable polymers, or a combination of biodegradable and biostable polymers.

The stent 100 is fabricated from a polymeric tube 200 shown in FIG. 2. The tube 200 may serves as a stent precursor construct in the sense that further processing may be performed on the tube before the pattern of stent struts is cut formed from the tube. The tube 200 is cylindrically-shaped with an outside diameter 205, an inside diameter 210, an outside surface 215, and a central axis 220. The tube 200 may be formed by various types of methods, including, but not limited to extrusion, injection molding, and rolling a flat sheet of material to form a tube. A pattern of struts may be formed on the tube 200 by chemical etching, mechanical cutting, and laser cutting material away from the tube. Representative examples of lasers that may be used include, but are not limited to, excimer, carbon dioxide, and YAG.

In some embodiments, the polymer tube 200 can have a outer diameter of 1-4 mm. The present invention is also applicable to polymer tubes less than 1 mm or greater than 4 mm in diameter. The wall thickness of the polymer tube can be between 0.1 mm to 0.3 mm. The present invention is also applicable to wall thicknesses below 0. 1 mm and above 0.3 mm.

As indicated above, the tube 200 may be formed by an extrusion process. During extrusion, a polymer melt is conveyed through an extruder which is then formed into a tube. Extrusion tends to impart large forces on the polymer molecules in the longitudinal direction of the tube due to shear forces on the polymer melt. The shear forces arise from forcing the polymer melt through an opening of a die at the end of an extruder. Additional shear forces may arise from any pulling and forming of the polymer melt upon exiting the die, such as may be performed in order to bring the extruded material to the desired dimensions of a finished tube. As a result, polymer tubes formed by some extrusion methods tend to possess a significant degree of molecular or crystal orientation in the direction that the polymer is extruded with a relatively low degree of orientation in the circumferential direction.

The degree of pulling that is applied to the polymer melt as it exits a die of an extruder and, thus, the degree of longitudinal orientation induced in the finished tube 200 can be partially characterized by what is referred to as a “draw down ratio.” Typically, the polymer melt is in the form of an annular film as it is extruded through and exits an annular opening of the die. The annular film has an initial outer diameter upon exiting the annular opening. The annular film is drawn or pulled, which causes a reduction of the annular film cross-sectional size to the final outer diameter. The drawn down portion of the tube may be cooled to ensure that it maintains its shape and diameter. The final outer diameter corresponds to the outer diameter of the finished, solidified polymeric tube 200. The draw down ratio is defined as the ratio of the final outer diameter to the initial outer diameter.

As indicated above, the finished, solidified polymeric tube 200 may serve as a precursor construct in that further processing of the tube is performed. Further processing includes heating combined with deformation of the tube in radial and axial directions, such as may be performed by blow molding. After blow molding, pieces of the blow molded tube are cut away to form stent struts.

The degree of radial expansion that the polymer tube undergoes can partially characterize the degree of induced circumferential molecular or crystal orientation as well as strength of the deformed tube in a circumferential direction. The degree of radial expansion is quantified by a radial expansion (“RE”) ratio, defined as RE Ratio=(Inside Diameter of Expanded Tube)/(Original Inside Diameter of the tube). The RE ratio can also be expressed as a percentage, defined as RE %=(RE ratio−1)×100%.

The degree of axial extension that the polymer tube undergoes can partially characterize induced axial molecular or crystal orientation as well as strength of the deformed tube in an axial direction. The degree of axial extension is quantified by an axial extension (“AE”) ratio, defined as AE Ratio=(Length of Extended Tube)/(Original Length of the Tube). The AE ratio can also be expressed as a percentage, defined as AE %=(AE ratio−1)×100%.

Blow molding includes first positioning the tube 200 in a hollow cylindrical member or mold. The mold controls the degree of radial deformation of the polymer tube by limiting the deformation of the outside diameter or surface of the polymer tube to the inside diameter of the mold. The inside diameter of the mold may correspond to a diameter less than or equal to a desired diameter of the finished polymer tube.

While in the mold, the temperature of the polymer tube 200 is heated to a temperature above Tg of the polymer to facilitate deformation. The temperature to which the tube 200 is heated during blow molding is a processing parameter referred to as the “expansion temperature” or “process temperature.” The heating of the polymer tube 200 to the expansion temperature can be achieved by heating a gas to the expansion temperature and discharging the heated gas onto an exterior surface of the mold containing the polymer tube.

While in the mold, one end of the polymer tube 200 is sealed or blocked. Thus, introduction of gas into the opposite end of the polymer tube will increase internal fluid pressure relative to ambient pressure in a region between the outer surface of the polymer tube and the inner surface of the mold. The internal fluid pressure is a processing parameter referred to as the “expansion pressure” or “process pressure.” Examples of gas that may be used to create the expansion pressure include without limitation ambient air, substantially pure oxygen, substantially pure nitrogen, and other substantially pure inert gases. In combination with other blow molding process parameters, the expansion pressure affects the rate at which the tube deforms radially and axially.

Blow molding may include pulling one end of the polymer tube 200. A tensile force, which is another processing parameter, is applied to one end of the polymer tube 200 while holding the other end of the polymer tube stationary. Alternatively, the two opposite ends of the polymer tube may be pulled apart. In combination with other blow molding process parameters, the tensile force affects the rate at which the tube deforms radially and axially.

The radially and axially deformed polymer tube may then be cooled from above Tg to below Tg, either before or after decreasing the pressure and/or decreasing tension. Cooling the deformed tube helps insure that the tube maintains the proper shape, size, and length following radial expansion and axial extension. The rate at which the deformed tube is cooled is yet another processing parameter. Slow cooling through a temperature range between Tm and Tg might result in a loss of amorphous chain orientation and cause a decrease in fracture toughness of the finished stent. Preferably, though not necessarily, the tube can be cooled quickly or quenched in relatively cold gas or liquid to a temperature below Tg to maintain chain orientation that was formed during tubing expansion.

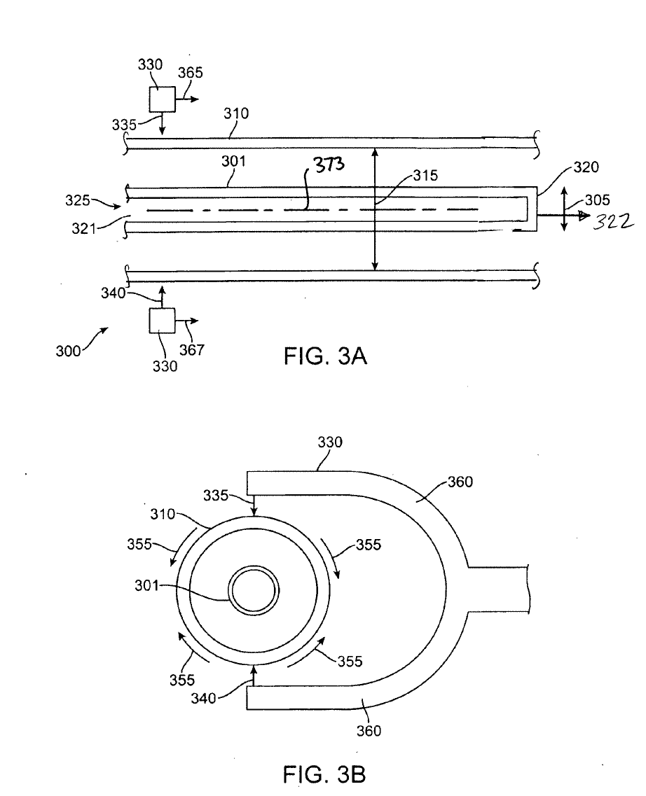

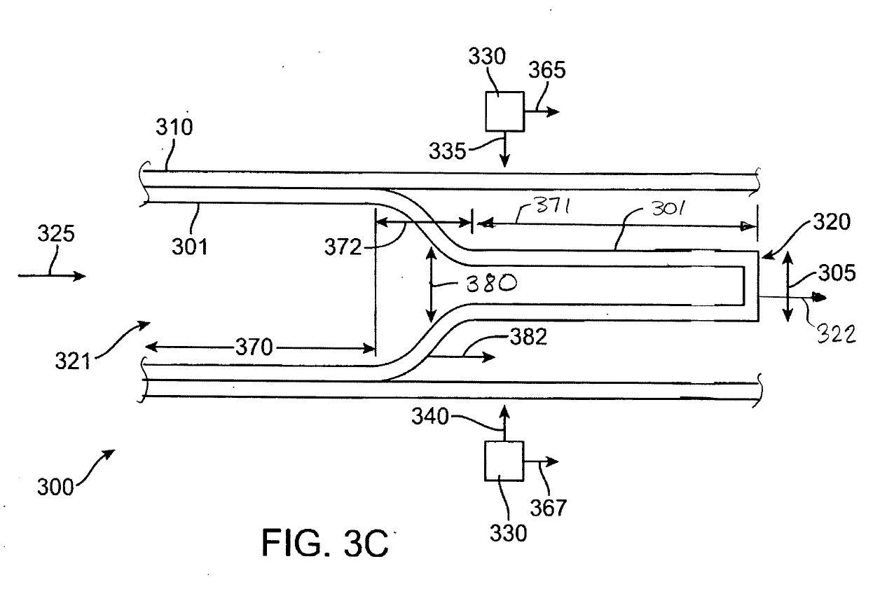

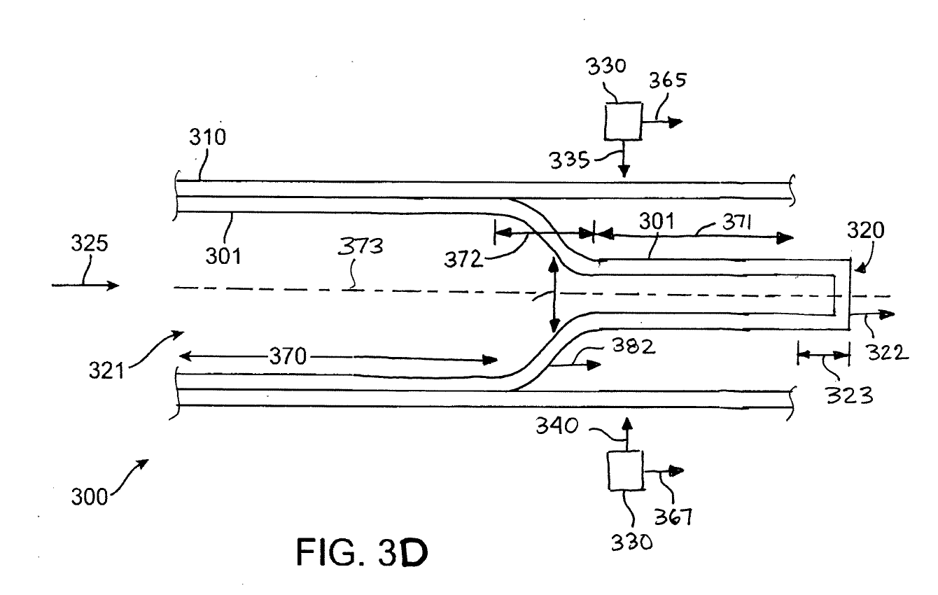

FIGS. 3A-D schematically depicts a molding system 300 for simultaneous radial and axial deformation of a polymer tube. FIG. 3A depicts an axial cross-section of a polymer tube 301 with an undeformed outside diameter 305 positioned within a mold 310. The mold 310 limits the radial deformation of the polymer tube 301 to a diameter 315 corresponding to the inside diameter of the mold 310. The polymer tube 301 is closed at a distal end 320. A gas is conveyed, as indicated by an arrow 325, into an open end 321 of the polymer tube 301 to increase internal fluid pressure within tube 301.

A tensile force 322 is applied to the distal end 320 in an axial direction. In other embodiments, a tensile force is applied at the proximal end 321 and the distal end 320.

A circular band or segment of the polymer tube 300 is heated by a nozzle 330. The nozzle has fluid ports that direct a heated fluid, such as hot air, at two circumferential locations of the mold 310, as shown by arrows 335 and 340. FIG. 3B depicts a radial cross-section showing the tube 301 within the mold 310, and the nozzle 330 supported by structural members 360. Additional fluid ports can be positioned at other circumferential locations of the mold 310 to facilitate uniform heating around a circumference of the mold 310 and the tube 301. The heated fluid flows around the mold 310, as shown by arrows 355, to heat the mold 310 and the tube 301 to a predetermined temperature above ambient temperature.

The nozzle 330 translates along the longitudinal axis 373 of the mold 310 as shown by arrows 365 and 367. That is, the nozzle 330 moves linearly in a direction parallel to the longitudinal axis 373 of the mold 310. As the nozzle 330 translates along the axis of the mold 310, the tube 301 radially deforms. The combination of elevated temperature of the tube 301, the applied axial tension, and the applied internal pressure cause simultaneous axial and radial deformation of the tube 301, as depicted in FIGS. 3C and 3D.

FIG. 3C depicts the system 300 with an undeformed section 371, a deforming section 372, and a deformed section 370 of the polymer tube 301. Each section 370, 371, 372 is circular in the sense that each section extends completely around the central axis 373. The deforming section 372 is in the process of deforming in a radial direction, as shown by arrow 380, and in an axial direction, as shown by arrow 382. The deformed section 370 has already been deformed and has an outside diameter that is the same as the inside diameter of the mold 310.

FIG. 3D depicts the system 300 at some time period after FIG. 3C. The deforming section 372 in FIG. 3D is located over a portion of what was an undeformed section in FIG. 3C. Also, the deformed section 370 in FIG. 3D is located over what was the deforming section 372 in FIG. 3C. Thus it will be appreciated that the deforming section 372 propagates linearly along the longitudinal axis 373 in the same general direction 365, 367 that the heat sources 330 are moving.

In FIG. 3D, the deforming section 372 has propagated or shifted by an axial distance 374 from its former position in FIG. 2D. The deformed section 370 has grown longer by the same axial distance 374. Deformation of the tube 301 occurs progressively at a selected longitudinal rate along the longitudinal axis 373 of the tube. Also, the tube 301 has increased in length by a distance 323 compared to FIG. 3C.

Depending on other processing parameters, the speed at which the heat sources or nozzles 330 are linearly translated over the mold 310 may correspond to the longitudinal rate of propagation (also referred to as the axial propagation rate) of the polymer tube 301. Thus, the distance 374 that the heat sources 330 have moved is the same distance 375 that the deformed section 370 has lengthened.

The rate or speed at which the nozzles 330 are linearly translated over the mold 310 is a processing parameter that relates to the amount of time a segment of the polymer tube is heated at the expansion temperature and the uniformity of such heating in the polymer tube segment.

It is to be understood that the tensile force, expansion temperature, and expansion pressure are applied simultaneously to the tube 301 while the nozzle 330 moves linearly at a constant speed over the mold. Again, the “expansion pressure” is the internal fluid pressure in the polymer tube while it is blow molded inside the mold. In FIGS. 3A-3D, the “expansion temperature” is the temperature to which a limited segment of the polymer tube is heated during blow molding. The “limited segment” is the segment of the polymer tube surrounded by the nozzle 330. The “limited segment” may include the deforming section 372. The heating of the polymer tube to the expansion temperature can be achieved by heating a gas to the expansion temperature and discharging the heated gas from the nozzle 330 onto the mold 310 containing the polymer tube.

The processing parameters of the above-described blow molding process include without limitation the tensile force, expansion temperature, the expansion pressure, and nozzle translation rate or linear movement speed. It is expected that the rate at which the tube deforms during blow molding depends at least upon these parameters. The deformation rate has both a radial component, indicated by arrow 380 in FIGS. 3C and 3D, and an axial component, indicated by an arrow 382. It is believed that the radial deformation rate has a greater dependence on the expansion pressure and the axial component has a greater dependence on the translation rate of the heat source along the axis of the tube. It is also expected that the deformation rate is dependant upon the pre-existing morphology of the polymer in the undeformed section 371. Also, since deformation rate is a time dependent process, it is expected to have an effect on the resulting polymer morphology of the deformed tube after blow molding.

The term “morphology” refers to the microstructure of the polymer which maybe characterized, at least in part, by the percent crystallinity of the polymer, the relative size of crystals in the polymer, the degree of uniformity in spatial distribution of crystals in the polymer, and the degree of long rage order or preferred orientation of molecules and/or crystals. The crystallinity percentage refers to the proportion of crystalline regions to amorphous regions in the polymer. Polymer crystals can vary in size and are sometimes geometrically arranged around a nucleus, and such arrangement may be with or without a preferred directional orientation. A polymer crystal may grow outwardly from the nucleus as additional polymer molecules join the ordered arrangement of polymer molecule chains. Such growth may occur along a preferred directional orientation.

Applicant believes that all the above-described processing parameters affect the morphology of the deformed polymer tube 301. As used herein, “deformed tube 301” and “blow molded tube 301” are used interchangeably and refer to the deformed section 370 of the polymer tube 301 of FIGS. 3C and 3D. Without being limited to a particular theory, Applicant believes that increasing the crystallinity percentage will increase the strength of the polymer but also tends to make the polymer brittle and prone to fracture when the crystallinity percentage reaches a certain level. Without being limited to a particular theory, Applicant believes that having a polymer with relatively small crystal size has higher fracture toughness or resistance to fracture. Applicant also believes that having a deformed tube 301 with spatial uniformity in the radial direction, axial direction, and circumferential direction also improves strength and fracture toughness of the stent made from the deformed tube.

It should be noted that the above-described processing parameters are interdependent or coupled to each other. That is, selection of a particular level for one processing parameter affects selection of appropriate levels for the other processing parameters that would result in a combination of radial expansion, axial extension, and polymer morphology that produces a stent with improved functional characteristics such as reduced incidence of strut fractures and reduced recoil. For example, a change in expansion temperature may also change the expansion pressure and nozzle translation rate required to obtain improved stent functionality.

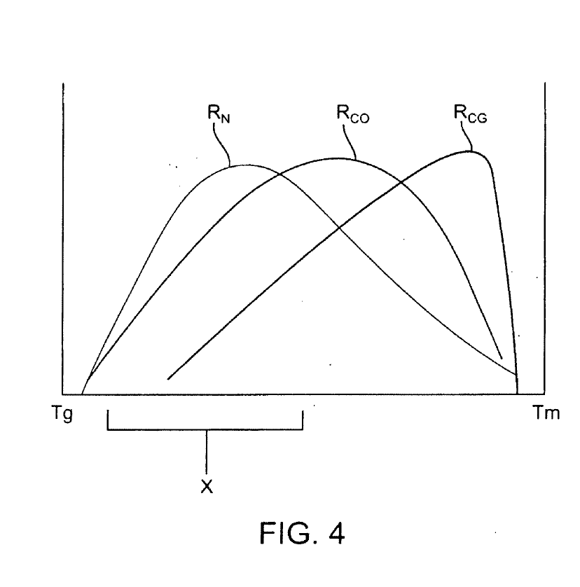

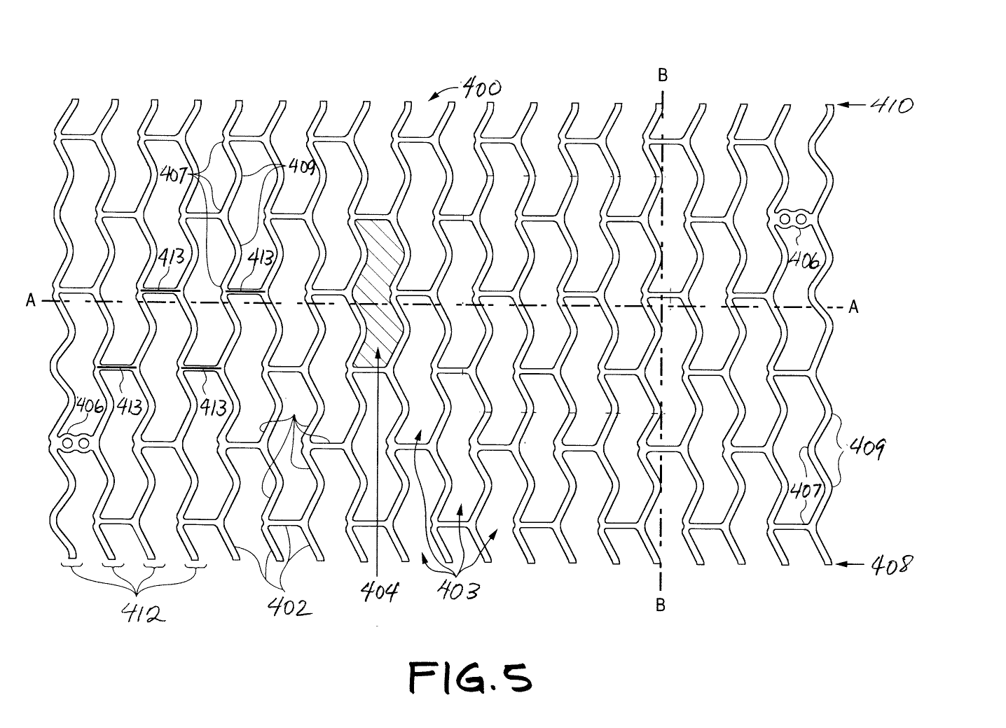

Expansion temperature affects the ability of the polymer to deform (radially and axially) while simultaneously influencing crystal nucleation rate and crystal growth rate, as shown in FIG. 4. FIG. 4 depicts an exemplary schematic plot of crystallization under quiescent condition, showing crystal nucleation rate (“R_{N}”) and the crystal growth rate (“R_{CG}”) as a function of temperature. The crystal nucleation rate is the rate at which new crystals are formed and the crystal growth rate is the rate of growth of formed crystals. The exemplary curves for R_{N} and R_{CG} in FIG. 4 have a curved bell-type shape that is similar to R_{N} and R_{CG} curves for PLLA. The overall rate of quiescent crystallization (“R_{CO}”) is the sum of curves R_{N} and R_{CG}.

Quiescent crystallization can occur from a polymer melt, which is to be distinguished from crystallization that occurs solely due to polymer deformation. In general, as shown in FIG. 4, quiescent crystallization tends to occur in a semi-crystalline polymer at temperatures between Tg and Tm of the polymer. The rate of quiescent crystallization in this range varies with temperature. Near Tg, nucleation rate is relatively high and quiescent crystal growth rate is relatively low; thus, the polymer will tend to form small crystals at these temperatures. Near Tm, nucleation rate is relatively low and quiescent crystal growth rate is relatively high; thus, the polymer will form large crystals at these temperatures.

As previously indicated, crystallization also occurs due to deformation of the polymer. Deformation stretches long polymer chains and sometimes results in fibrous crystals generally oriented in a particular direction. Deforming a polymer tube made of PLLA by blow molding at a particular expansion temperature above Tg results in a combination of deformation-induced crystallization and temperature-induce crystallization.

As indicated above, the ability of the polymer to deform is dependent on the blow molding temperature (“expansion temperature”) as well as being dependant on the applied internal pressure (“expansion pressure”) and tensile force. As temperature increases above Tg, molecular orientation is more easily induced with applied stress. Also, as temperature approaches Tm, quiescent crystal growth rate increases and quiescent nucleation rate decreases. Thus, it will also be appreciated that the above described blow molding process involves complex interaction of the processing parameters all of which simultaneously affect crystallinity percentage, crystal size, uniformity of crystal distribution, and preferred molecular or crystal orientation.

The desired mechanical properties of the stent made from the deformed tube 301 includes high radial strength, high toughness, high modulus, and low recoil upon deployment of the stent. High toughness can be demonstrated by a lower incidence of cracked and/or broken struts upon expansion of the stent to a deployment diameter.

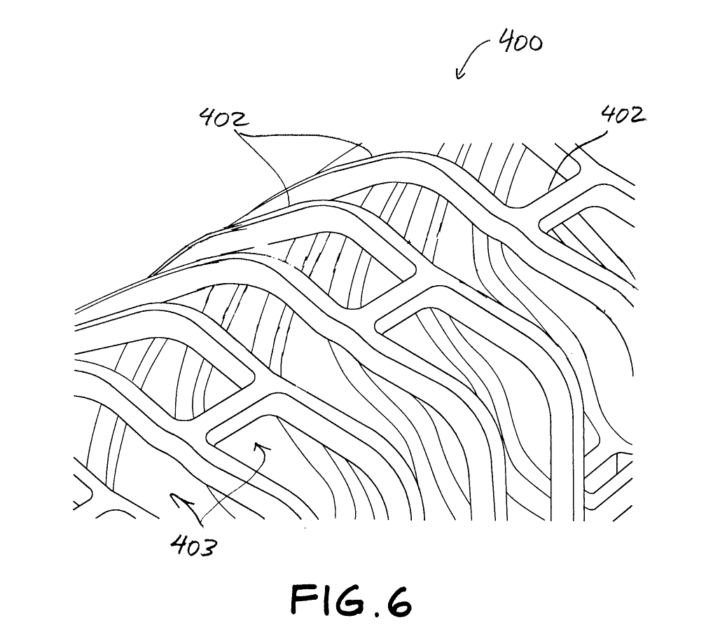

FIG. 5 shows another stent pattern 400 illustrated in a planar or flattened view for ease of illustration and clarity. The stent pattern 400 was cut from a tubular precursor construct. Thus, stent pattern 400 actually forms a tubular stent structure, as partially shown in FIG. 6, so that line A-A is parallel to the central axis of the stent. FIG. 6 shows the stent in a fully deployed state.

The stent pattern 400 includes various struts 402 oriented in different directions and gaps 403 between the struts. Each gap 403 and the struts 402 immediately surrounding the gap defines a closed cell 404. At the proximal and distal ends of the stent, a strut 406 includes depressions, blind holes, or through holes adapted to hold a radiopaque marker that allows the position of the stent inside of a patient to be determined. One of the closed cells 404 is shown with cross-hatch lines to illustrate the shape and size of the cells. All the cells 404 have the same size and shape.

The pattern 400 is illustrated with a bottom edge 408 and a top edge 410. On a stent, the bottom edge 408 meets the top edge 410 so that line B-B forms a circle around the stent central axis. In this way, the stent pattern 400 forms sinusoidal hoops or rings 412 that include a group of struts arranged circumferentially. The rings 412 include a series of crests 407 and troughs 409 that alternate with each other. The sinusoidal variation of the rings 412 occurs primarily in the axial direction, not in the radial direction. That is, all points on the outer surface of each ring 412 are at the same or substantially the same radial distance away from the central axis of the stent.

Still referring to FIG. 5, the rings 412 are connected to each other by another group of struts that have individual lengthwise axes 413 parallel or substantially parallel to line A-A. The rings 412 are capable of being collapsed to a smaller diameter during crimping and expanded to their original diameter or to a larger diameter during deployment in a vessel.

The present invention applies to any stent pattern, not just to the pattern shown in FIGS. 5 and 6. A stent may have a different number of rings 412 and cells 404 than what is shown. The number and size of rings 412 and cells 404 may vary depending on the desired axial length and the desired deployed diameter of the stent. For example, a diseased segment of a vessel may be relatively small so a stent having a fewer number of rings can be used to treat the diseased segment.

Applicant has unexpectedly found that stents cut from a PLLA tube that has been blow molded under certain processing parameter levels demonstrate improved fracture toughness upon deployment while maintaining sufficient flexibility for crimping and delivery and sufficient radial strength to prevent undue recoil. The PLLA tube was made entirely of PLLA. The preferred levels are given below for the blow molding process parameters for a PLLA precursor tubular construct having an initial (before blow molding) crystallinity percentage of up to about 20% and more narrowly from about 5% to about 15%. Applicant believes that the blow molding process parameter levels given blow result in a deformed PLLA tube having a crystallinity percentage below 50% and more narrowly from about 30% to about 40%.

In combination with other blow molding process parameters, improved performance in PLLA stents was seen with percent radial expansion (RE %) from about 200% to about 600%, and more narrowly from about 300% to about 500%, and more narrowly at or about 400%. In combination with other blow molding process parameters, Applicant found that when RE % exceeded 600%, there was no significant increase in radial strength while more cracks were found along the axial direction of the stent as a result of use, especially in stents that have aged prior to use. In combination with other blow molding process parameters, Applicant found that when RE % is about 100% or less, the radial strength was too low for a stent having a strut thickness of 0.006 inches, making the stent highly susceptible to fracture during crimping, delivery, and deployment.

TABLE I shows the effect of radial expansion on stent functional performance as measured by the number of cracks or broken struts. The stents that were tested had the strut pattern of FIG. 5. There were four groups of stents tested. Each group of stents were made from a precursor construct made of PLLA (“PLLA tube”) that had been deformed radially and axially by blow molding. For each group, stents cut from the deformed PLLA tubes were expanded from a crimped state to a deployed (expanded) diameter to simulate what occurs during implantation in a patient. The number of stents with at least one broken struts and the number of strut cracks per stent were noted for deployed diameters of 3.0 mm, 3.5 mm and 4.0 mm. A strut was counted as broken when a crack propagated all the way through the strut. A size criteria was used when counting cracks that did not go all the way through the strut: only cracks that propagated at least 50% of the strut width were counted. Thus, TABLE I shows that for stents made from a 300% radially expanded PLLA tube then deployed to 3.0 mm, the number of cracks satisfying the size criteria ranged from 2 cracks per stent to 39 cracks per stent. For stents made from a 500% radially expanded PLLA tube then deployed to 4.0 mm, three stents exhibited broken struts and the number of cracks satisfying the size criteria ranged from 9 per stent to 30 per stent.

TABLE I

| | | | | | Stent deployed to | |
|---|---|---|---|---|---|---|
| | Stent deployed to | | Stent deployed to | | 4.0 mm diameter | |
| Radial | 3.0 mm diameter | | 3.5 mm diameter | | | # of |
| Expansion | # stents | # of | # stents | # of | # stents | cracks |
| of | with | cracks | with | cracks | with | per |
| Precursor | broken | per stent | broken | per stent | broken | stent |
| Construct | struts | (note 1) | struts | (note 1) | struts | (note 1) |
| 300% | 0 | 2 to 39 | 0 | 2 to 22 | 6 | 1 to 17 |
| 400% | 0 | 0 | 0 | 0 | 0 | 0 |
| 450% | 0 | 0 to 8 | 0 | 0 to 13 | 0 | 1 to 6 |
| 500% | 1 | 1 to 23 | 1 | 18 to 37 | 3 | 9 to 30 |

(note 1) Number of cracks having a size that is at least 50% of the strut width, per stent.

TABLE I shows that stents cut from PLLA tubes that were radially expanded to 400% performed best, as this group exhibited no broken struts and no cracks after being deployed, whether deployed to a diameter of 3.0 mm, 3.5 mm, or 4.0 mm. “No cracks” means that there were no cracks of a size that was at least 50% of the strut width. By contrast, radial expansion below 400% (to 300%) and above 400% (to 450% and 500%) resulted in cracks greater than 50% of strut width. Broken struts occurred with radial expansion of 300% and 500%.

When the number of broken struts is weighted more than the number cracks, the column with the worst performance corresponds to stents deployed to 4.0 mm diameter. Notably within in this column, stents formed from PLLA tubes radially expanded to 400% exhibited no broken struts and no cracks of a size greater than 50% of strut width.

We turn now to the axial extension processing parameter. In combination with other blow molding process parameters, improved performance in PLLA stents was seen with percent axial extension (AE %) from about 10% to about 400%, and more narrowly from about 20% to about 200%, and more narrowly from about 20% to about 70%, and more narrowly at about 20%. In combination with other blow molding process parameters, Applicant found that when AE % is about 100% or more, the stent exhibited more cracks and broken struts along the circumferential direction during stent deployment.

The selected level for AE % may depend on the degree of axial orientation that is already present in an extruded tube that is used as the polymer precursor construct. As previously indicated, a significant amount of axial orientation may already be induced in the precursor construct as a result of extrusion and draw down. In combination with other blow molding process parameters, Applicant has unexpectedly found improved stent functionality when the stent is formed from an extruded tube subjected to AE % of about 20% to 70% during blow molding, wherein prior to blow molding the tube extrusion process used a draw down ratio in the range of about 8:1 to about 2:1, more narrowly from about 7:1 to about 3:1, and more narrowly about 7:1.

As previously indicated, the stent is subject to deformation during stent deployment. Some portions of the stent are stretched while other portions of the stent are compressed. Deformation during stent deployment is believed to occur mostly in the circumferential direction, though some deformation also occurs in the axial direction and in directions other than axial and circumferential. Therefore, Applicant believes that at least some axial orientation of polymer molecule chains is desirable. In one study, axial extension of the precursor construct was varied from 0% to 300%. Many cracks and broken struts were observed after deployment of stents made from a precursor construct that was axially expanded above 100%. Above 100%, the incidence of cracks and broken struts generally increased proportionally with greater axial extension. A lower incidence of cracks and broken struts was observed with axial extension in the range of about 20% to about 70%.

We turn next to the tensile force processing parameter. In combination with other blow molding process parameters, improved performance in PLLA stents was seen with a tensile force corresponding to about 84 grams applied to one end of the tube during blow molding.

We turn now to the propagation rate processing parameter, which corresponds to the rate at which a deforming section of the polymer tube travels along the length of the polymer tube, and may also correspond to the rate at which heating nozzles are linearly translated across the mold. In combination with other blow molding process parameters, improved performance in PLLA stents was seen with an axial propagation rate no greater than about 0.3 mm/minute compared to rates from about 0.6 mm/minute to about 2 mm/minute.

In combination with other blow molding process parameters, improved performance in PLLA stents was seen with an expansion pressure in the tubular construct in the blow mold at a gauge pressure of about 130 pounds per square inch (psi) or less, and more narrowly in the range of about 110 psi to about 130 psi. In combination with the other blow molding process parameters, an expansion pressure below 70 psi is often insufficient to expand the polymer tube, while an expansion pressure above 180 psi may produce air bubbles in the polymer. Air bubbles are believed to increase the incidence of broken struts and cracks.

Next we turn to the expansion temperature processing parameter. In combination with other blow molding process parameters, improved performance in PLLA stents was seen with an expansion temperature between about 160 deg F. to about 220 deg F., and more narrowly between about 160 deg F. and 190 deg F., and more narrowly between about 170 deg F. and about 180 deg F., and more narrowly at about 170 deg F.

In some embodiments, the expansion temperature is at a selected level above Tg of the polymer of the tubular construct in the blow mold. As with other polymers, Tg for PLLA may vary depending on the processing history of the polymer. For PLLA, Tg may range from 122 deg F. to 176 deg F. (50 deg. C. to 80 deg. C.) and, more narrowly, between about 136 deg F. to about 140 deg F. (58 deg. C. to about 60 deg. C.). In combination with other blow molding process parameters, improved performance in PLLA stents was seen with an expansion temperature that is between 20 to 50 deg. C. above Tg, and more narrowly at or about 20 deg. C. above Tg.

A precursor construct may also be made from other polymers, such as poly(lactic-co-glycolic acid) (“PLGA”). PLGA is a copolymer of LLA and GA. When the proportion of GA is increased, the maximum crystallinity of PLGA decreases and the degradation rate increases. Different forms of PLGA may be used in a precursor construct for a stent. The different forms may be identified with regard to the selected monomer ratio. The precursor construct can be made from PLGA including any molar ratio of L-lactide (LLA) to glycolide (GA). For example, without limitation, the precursor construct can be made from PLGA with a molar ratio of (LA:GA) including 85:15 (or a range of 82:18 to 88:12), 95:5 (or a range of 93:7 to 97:3), or commercially available PLGA products identified as having these molar ratios. Tg for various forms of PLGA ranges from about 104 deg F. to 140 deg F. (40 deg C. to 60 deg. C.).

For PLGA with a molar ratio (LA:GA) of 85:15, Tm is about 40 deg. C. lower than that of PLLA, so PLGA 85:15 can be extruded to form a precursor tube at about 20 deg. C. to about 40 deg. C. lower than the extrusion temperature for PLLA. Also, Tg for PLGA 85:15 is about 10 deg. C. lower than that of PLLA, so a precursor tube made of PLGA 85:15 can normally be expanded at a relatively low lower expansion pressure (i.e., process pressure) of about 110 psi. For PLGA 85:15, an axial propagation rate no greater than about 0.3 mm/minute is preferred. The axial propagation rate corresponds to the speed at which heat sources or nozzles are linearly translated over a blow mold containing the precursor tube.

It is contemplated that alternative polymers formulations, such as PLLA-based bioabsorbable copolymers or blends containing rigid and soft segments, might have less stiffness and better toughness. Examples for the rigid segment include without limitation PLA and PLGA. Examples for the soft segment include without limitation polycaprolactone (“PCL”) and polytrimethylcarbonate (“PTMC”). An example of a PLLA-based bioabsorbable copolymer containing rigid and soft segments is, without limitation, poly(L-lactide-co-caprolactone) copolymer. An examples of a PLLA-based bioabsorbable blend containing rigid and soft segments is poly poly(L-lactide)/poly(L-lactide)-block-polycaprolactone. A precursor tube made from any one or a combination of these alternative polymer formulations may be processed in the same manner as described above for a PLLA precursor tube. For example, and not limitation, expansion temperature during blow molding can be between 20 to 50 deg. C. above Tg, and more narrowly at or about 20 deg. C. above Tg of the polymer formulation. Deformation of a precursor tube made from any one or a combination of these alternative polymer formulations can involve any one or any combination of the following process steps:

(a) maintaining fluid pressure in the precursor tube at a process pressure from about 50 psi to about 200 psi, or more narrowly in the range of about 75 psi to about 175 psi, or more narrowly in the range of about 100 psi to about 150 psi, or in the range of about 110 psi to about 130 psi, or in the range of about 50 psi to about 75 psi, or in the range of about 75 psi to about 100 psi, or in the range of about 100 psi to about 125 psi, or in the range of about 125 psi to about 150 psi, or in the range of about 150 psi to about 175 psi, or in the range of about 175 psi to about 200 psi;

(b) heating the precursor tube to a process temperature from about 100 deg F. to about 300 deg F., more narrowly in the range of about 125 deg F. to about 275 deg F., or in the range of about 150 deg F. to about 250 deg F., or in the range of about 160 deg F. to about 220 deg F., or in the range of about 100 deg F. to about 150 deg F., or in the range of about 150 deg F. to about 200 deg F., or in the range of about 200 deg F. to about 250 deg. F, or in the range of about 250 deg F. to about 300 deg F.;

(c) radially expanding the precursor tube during the maintaining of fluid pressure and the heating, the radial expansion being according to a radial expansion ratio between about 100% and about 600%, or in the range of about 150% to about 550%, or in the range of about 200% to about 500%, or in the range of about 250% to about 500%, or in the range of about 300% to about 450%, or in the range of about 100% to about 200%, or in the range of about 200% to about 300%, or in the range of about 300% to about 400%, or in the range of about 400% to about 500%, or in the range of about 500% to about 600%;

(d) axially extending the precursor tube during the maintaining of fluid pressure and the heating, the axial extension being according to an axial extension ratio from about 10% to about 200%, or from about 15% to about 150%, or from about 18% to about 120%, or from about 20% to about 100%, or in the range of about 10% to about 50%, or in the range of about 50% to 100%, or in the range of about 100% to about 150%, or in the range of about 150% to about 200%;

(e) heating the precursor tube may include heating a tubular mold containing the precursor tube, the heating including moving a heat source disposed outside the precursor tube at a linear rate of movement parallel to the central axis of the mold, the linear rate of movement being from about 0.05 mm per minute to about 1.5 mm per minute, or from about 0.07 mm per minute to about 1.0 mm per minute, or from about 0. 1 mm per minute to about 0.7 mm per minute, or in the range of about 0.1 mm per minute to about 0.3 mm per minute, or in the range of about 0.3 mm per minute to about 0.6 mm per minute; and

(f) heating the precursor tube may further include applying a load to an end of the precursor tube during the maintaining of fluid pressure and the heating, the load being from about 20 grams to 200 grams, or from about 40 grams to about 175 grams, or from about 50 grams to about 150 grams, or in the range of about 20 grams to about 50 grams, or in the range of about 50 grams to about 100 grams, or in the range of about 100 grams to about 150 grams, or in the range of about 150 grams to about 200 grams.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

What is claimed is:

1. A method for making a stent, the method comprising: deforming a precursor tube of poly(L-lactide) to form a deformed tube, the deforming including:
maintaining fluid pressure in the precursor tube at a process pressure from about 110 psi to about 150 psi,
heating the precursor tube to a process temperature from about 160 deg F. to about 220 deg F.,
radially expanding the precursor tube according to a radial expansion ratio between about 300% and about 450% during the maintaining of fluid pressure and the heating, and
axially extending the precursor tube according to an axial extension ratio from about 20% to about 100% during the maintaining of fluid pressure and the heating; and
forming a network of stent struts from the deformed tube.

2. The method of claim 1, wherein heating the precursor tube includes heating a tubular mold containing the precursor tube, the heating including moving a heat source disposed outside the precursor tube at a linear rate of movement parallel to the central axis of the mold, the linear rate of movement being from about 0.1 mm per minute to about 0.7 mm per minute.

3. The method of claim 2, wherein the linear rate of movement is about 0.3 mm per minute.

4. The method of claim 1, wherein deforming further includes applying a load from about 50 grams to about 150 grams to an end of the precursor tube during the maintaining of fluid pressure and the heating.

5. The method of claim 1, wherein the deformed tube has a crystallinity from about 30% to about 50%.

6. The method of claim 1, wherein the precursor tube has a crystallinity from about 5% to about 15%.

7. The method of claim 1, wherein the precursor tube is an extrusion of poly(L-lactide).

8. The method of claim 1, further comprising extruding poly(L-lactide) to form the precursor tube, the extruding including a draw-down ratio from about 7:1 to about 3:1.

9. The method of claim 1, wherein the radial expansion ratio is about 400%.

10. The method of claim 1, wherein the process temperature is from about 170 deg F. to about 180 deg F.

11. A stent comprising a network of stent struts formed according to the method of claim 1.

12. A method of making a stent, the method comprising:
providing a poly(L-lactide) tube inside a tubular mold;
heating a segment of the tube with a heat source, the segment of the tube being heated to a process temperature from about 160 deg F. to about 220 deg F.;
moving the heat source in a process direction;
causing deformation of the heated segment to form a deformed segment of the tube, the deformation propagating in the process direction, the deformation including radial expansion and axial extension of the tube, the radial expansion in accordance with a radial expansion ratio between about 300% and about 450%, the axial extension in accordance with an axial extension ratio between about 20% and about 100%; and
forming stent struts from the deformed segment.

13. The method of claim 12, wherein the deformation propagates in the process direction at about 0.3 mm per minute.

14. The method of claim 12, wherein causing deformation of the heated segment includes maintaining fluid inside the tube at a pressure from about 110 psi to about 150 psi.

15. The method of claim 14, wherein the heat source moves in the process direction at about 0.3 mm per minute.

16. The method of claim 15, wherein the process temperature is from about 170 deg. F. to about 180 deg F.

17. The method of claim 16, wherein the radial expansion ratio is about 400%.

18. A method for making a stent, the method comprising:
deforming a precursor tube of a polymer formulation to form a deformed tube, the deforming including:
maintaining fluid pressure in the tube at a process pressure from about 50 psi to about 200 psi,
heating the tube to a process temperature from about 100 deg F. to about 300 deg F.,
radially expanding the precursor tube according to a radial expansion ratio between about 100% and about 600% during the maintaining of fluid pressure and the heating, and
axially extending the precursor tube according to an axial extension ratio from about 10% to about 200% during the maintaining of fluid pressure and the heating; and
forming a network of stent struts from the deformed tube.

19. The method of claim 18, wherein the precursor tube is an extrusion of the polymer formulation, and the polymer formulation is selected from the group consisting of PLGA, PLLA-co-PDLA, PLLD/PDLA stereocomplex, and PLLA-based polyester block copolymer containing a rigid segment and a soft segment, the rigid segment being PLLA or PLGA, the soft segment being PCL or PTMC.

20. A method of making a stent, the method comprising:
providing a polymer tube inside a tubular mold, the polymer tube made of a polymer formulation selected from the group consisting of PLGA, PLLA-co-PDLA, PLLD/PDLA stereocomplex, and PLLA-based polyester block copolymer containing a rigid segment and a soft segment, the rigid segment being PLLA or PLGA, the soft segment being PCL or PTMC;
heating a segment of the tube with a heat source, the segment of the tube being heated to a process temperature from about 100 deg F. to about 300 deg F.;
moving the heat source in a process direction;
causing deformation of the heated segment to form a deformed segment of the tube, the deformation propagating in the process direction, the deformation including radial expansion and axial extension of the tube, the radial expansion in accordance with a radial expansion ratio between about 100% and about 600%, the axial extension in accordance with an axial extension ratio from about 10% to about 200%; and
forming stent struts from the deformed segment.
